(19) United States
(12) Patent Application Publication
(10) Pub. No.: US 2026/0090805 A1
(43) Pub. Date: Apr. 2, 2026

(54) STRUCTURED, SUPPORTED WOUND CLOSURE

(71) Applicant: DermaClip US, LLC, Houston, TX (US)

(72) Inventors: Charles M. Darling, IV, Houston, TX (US); Catherine M. Fonfara, Houston, TX (US); Christopher B. Darling, New York, NY (US)

(73) Assignee: DermaClip US, LLC, Houston, TX (US)

(21) Appl. No.: 19/196,157

(22) Filed: May 1, 2025

Related U.S. Application Data
(60) Provisional application No. 63/641,185, filed on May 1, 2024.

(51) Int. Cl. A61B 17/08

(52) U.S. Cl. CPC A61B 17/085; A61B 2017/081; A61B 2017/088

(57) ABSTRACT

A wound closure device with a structure that reliably induces eversion of tissue edges and fully supports the edges that define a wound opening.

### REFERENCE TO RELATED APPLICATIONS

The present claims priority to Provisional U.S. Application No. 63/641,185, filed May 1, 2024, all entitled “STRUCTURED, SUPPORTED WOUND CLOSURE,” which is incorporated in its entirety.

### TECHNICAL FIELD

This invention is directed to tissue closure devices, tools, systems, kits, and methods for tissue repair and closure. In particular, the invention is directed to devices and methods that safely and securely close wounded tissue openings and permit and encourage healing to occur.

### BACKGROUND ART

Closure of tissue openings, such as, for example, for surgical incisions and accidental lacerations or wounds, is critical both to minimize the risk of infection and to promote optimal healing of the wound or incision. Both of these outcomes require rapid wound closure and careful skin edge approximation. Closing a tissue opening or wound requires a mechanism for drawing both sides of a tissue opening together to promote healing and to reduce the formation of scar tissue.

Previous wound closure systems included various categories of materials passed through the skin, such as staples and sutures, substances that cover skin edges and hold them adjacent, such as glues, and adherent structures, such as strips. Suturing and stapling and tape procedures are invasive, which can traumatize and compromise the integrity of the tissue opening and the nutrient blood supply to the healing tissue edges. They cause pain, increase the possibility of infection, expose the surgeon, as well as the patient, to blood-borne disease, generally leave behind significant scars, and require a follow-up visit for suture or staple removal. Surgical glue is also used but, if unsupported by subcutaneous stitching, is generally only adequate for small wounds or incisions where skin edges are not widely separated or under tension during closure. Tape procedures are generally ineffective for larger wounds and are not typically used for a variety of reasons in most surgical procedures.

Typical techniques of suturing to minimize the resulting blemish that occurs during the healing process require a threshold of dexterity that many care providers do not possess. This is particularly true in emergency situations, which often require immediate treatment to secure the tissue opening to allow for transport or until such time as proper surgery is possible. Suturing even by a skilled surgeon punctures and stresses tissue causing scarring. An easy to use, suture-less tissue opening closure system would be a great benefit in many situations.

Scientific principles show that good cosmetic outcomes in wound healing are largely determined by apposition and eversion created during wound closure. Eversion is critically important as expected scar formation includes myofibril contraction, which ultimately draws the scar slightly below the surrounding skin level. This is often seen in surgical scars, which have divots or slight depressions in the center. Eversion prevents or at least minimizes this phenomenon by starting with slightly raised skin that lays flat after the universal process of contraction, which occurs at about 2-3 weeks after injury. The medical community generally recommends skin edge eversion to promote the least noticeable scar. Unfortunately, skin edge eversion and apposition is difficult to achieve with simple stitches or glues. More complex stitches and devices that promote adequate eversion and apposition are time consuming to place and are used in less than one case in one hundred. Additionally, conventional tissue closure devices for tissue do not allow for the breathing and the egress of tissue fluids. Often, the durability of the applied tissue device is compromised by the impervious barrier to moisture and other substances beneath polymer components. This may lead to dislodgement of the tissue device, wound maceration, or infection.

### SUMMARY OF THE EMBODIMENTS

The present invention overcomes the problems and disadvantages associated with current strategies and designs and provides new systems and methods of making devices for closing wounds.

A preferred embodiment of the invention is directed to a wound closure system. The system has a pair of identical closure components. Each closure component comprising a base, the base having an adhesive first side, a standoff assembly extending from a second side of the base, the standoff assembly having a pull-tab extending therefrom and a female portion adapted to accept the pull-tab of the other closure component, wherein a width of the base is equal to a width of the standoff assembly to provide support to the wound along the entire width of the closure components.

Preferably, upon engagement of the closure components, the components pivot about a contact point to evert the edges of the skin. Preferably, the base is comprised of a wing portion and an anchor portion and the standoff assembly is positioned above the wing portion. In a preferred embodiment, the wing portion and the standoff assembly are of a first width and the anchor portion is of a second, different width. The anchor portion is preferably narrower than the wing portion.

The system preferably further comprises a locking mechanism positioned within the female portion and at least one tooth extending from the pull-tab. The locking mechanism is preferably adapted to engage the at least one tooth of the pull-tab. Preferably, the locking mechanism is releasable. In a preferred embodiment, the locking mechanism is releasable via either a tool or a manipulatable finger.

The system is preferably adapted to exert a uniform pressure across the width of the closure components. The closure components are preferably adapted to prevent torsion of the wound. Preferably, each closure component is a single piece of material. In a preferred embodiment, each standoff assembly is comprised of a forward face from which the pull tab extends and a reward face, wherein the forward face is sloped rearward as it approaches the adhesive base. When a closure component is positioned on opposite sides of a wound and the pull tab of each closure component engages the opening in the standoff assembly of the opposing closure component, as the pull tabs are pulled away from each other, the wound is preferably closed such that the contact of the sloped forward faces of each standoff assembly cause eversion of tissue edges of the wound. The system is preferably non-invasive.

Other embodiments and advantages of the invention are set forth in part in the description, which follows, and in part, may be obvious from this description, or may be learned from the practice of the invention.

### BRIEF DESCRIPTION OF THE DRAWINGS

The foregoing features of embodiments will be more readily understood by reference to the following detailed description, taken with reference to the accompanying drawings, in which:

FIG. 1A shows an isometric view of an embodiment of a tissue closure device in the open position;

FIG. 1B shows another isometric view of the tissue closure device of FIG. 1A in the closed position.

FIG. 2A shows a top view of a closure component, in accordance with an embodiment of the invention;

FIG. 2B shows a side view of a closure component, in accordance with an embodiment of the invention;

FIG. 3 shows a cross-section of a device in the engaged position, in accordance with an embodiment of the invention;

FIG. 4 shows a cutaway isometric view of a closure component, in accordance with an embodiment of the invention;

FIG. 5 shows a cross-section of a device with a releasable locking mechanism in the engaged position, in accordance with an embodiment of the invention; and

FIGS. 6A-C show stages of closing a tissue closure device, in accordance with an embodiment of the invention.

### DETAILED DESCRIPTION OF SPECIFIC EMBODIMENTS

As embodied and broadly described herein, the disclosures herein provide detailed embodiments of the invention. However, the disclosed embodiments are merely exemplary of the invention that may be embodied in various and alternative forms. Therefore, there is no intent that specific structural and functional details should be limiting, but rather the intention is that they provide a basis for the claims and as a representative basis for teaching one skilled in the art to variously employ the present invention.

Definitions. As used in this description and the accompanying claims, the following terms shall have the meanings indicated, unless the context otherwise requires:

A “tissue opening” refers to any surgical incision, accidental laceration or other form of injury. A tissue opening may be a wound in the skin, or to a tissue within a living or dead body, such as an organ. The tissue opening may be associated with any animal, including human and non-human animals. In some embodiments, the tissue opening may be associated with a mammal or a non-human mammal. A tissue opening may be of any shape or size.

“Apposition” refers to the proper alignment of skin edges to prevent gaps or mismatch.

“Eversion” is the turning outward of the edges of a wound or incision in order to bring the live dermis on the underside of the outer layer of the skin into contact.

In illustrative embodiments of the invention, devices, tools, systems, kits and methods for closing a tissue opening non-invasively are presented. The devices include a tissue closure device that reliably induces eversion of tissue edges that define a tissue opening. Further embodiments may provide for releasable locking of the tissue closure devices, allowing for drainage of wound edema and/or treatment of infection, without removal and reapplication of the devices. Still further embodiments may allow for the passing of moisture or other substances that may collect under a tissue closure device.

An embodiment of the invention is directed to a device having two halves, each half composed of a polypropylene or other resin piece. The polypropylene piece is a unitary structure with the following elements along its continuance: an anchoring element; a living hinge connecting the anchoring element and the closure element; and the closure element. The entirety of the polypropylene piece has adhesive attached to it on its base side; the “base adhesive”; the top element of the adhesive sandwich is an adhesive attached to the top side of the anchoring element alone, stopping prior to the living hinge. In this manner an adhesive sandwich enclosing the anchoring element is created so as to securely bind the device to the skin. There is a pulling tab attached to the closure element of the polypropylene piece. This pulling tab is threaded through the closure element of the other polypropylene piece on the other half of the polypropylene piece so as to join the two halves together.

The base adhesive of each half of the device is placed on the skin with the outer edge of the closure element of the polypropylene piece attached to, and approximating, the edge of the incision or wound. In closure, the living hinge pivots so as to allow the closure device to pull the approximated edges of the wound held by each half of the joined device toward each other and then upward so as to cause eversion of the approximated edges of the skin upon closure. To effect closure, the pulling tabs attached to and part of the polypropylene piece are pulled away from each other so as to force closure of the device.

As to the anchoring element, holes have been put into the anchoring element so as to render it porous. The non-porous adhesive, combined with the porosity induced into the polypropylene, allows the wound to breath and exudate.

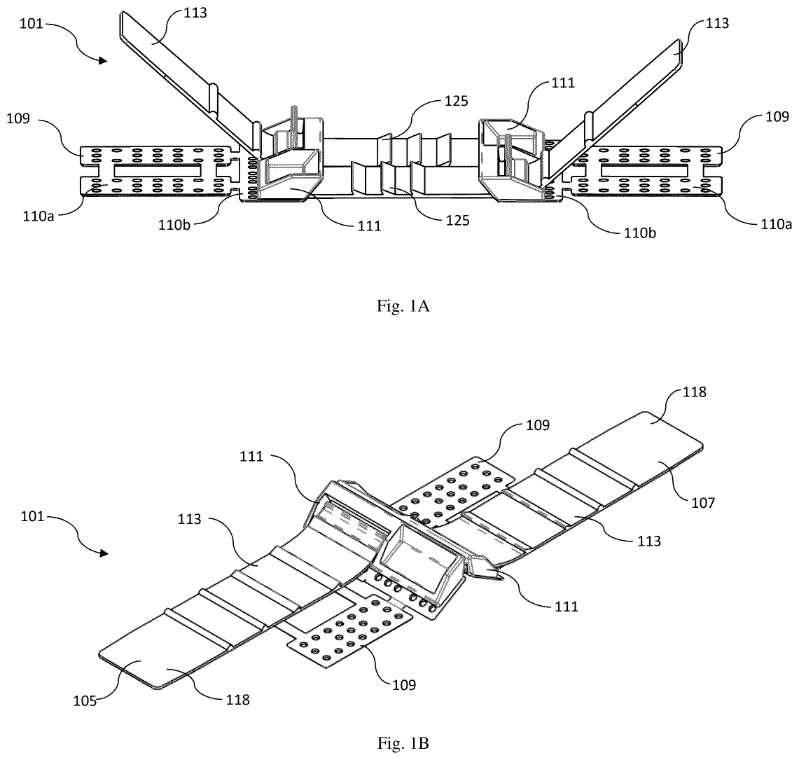

FIG. 1A shows an oblique view of a tissue closure device 101 applied to a tissue opening 103 prior to an operator effectuating tissue closure, while FIG. 1B shows an oblique view of the tissue closure device of FIG. 1A after the operator effectuates tissue closure, in accordance with various embodiments of the invention. The tissue closing device 101 may be made of various materials, including, without limitation, plastic, metal, polypropylene, high-density polypropylene, and other polymers or thermoplastic materials, or combinations thereof. In various embodiments, the materials used for the tissue closure device 101 will be light, strong, and/or waterproof. It is possible that with some materials, the tissue closure device 101 may be cleaned and sterilized for reuse. The tissue closure device 101 may be applied under sterile or medically clean conditions depending on the type of wound and according to the best judgment of the healthcare provider. In some embodiments, the tissue closure device 101 (or one or more members thereof) includes an agent, such as for example a chemical, to detect and signal impending infection or treat the wound. Alternatively, in some embodiments, such an agent may be added to or above the closure during or after the procedure. In some embodiments the wound closure components may be made of bioabsorbable materials, such as a polysaccharide material. Bioabsorbable materials may advantageously be used to close a tissue opening within a living body, whereupon extra surgery is not needed for removal of the tissue closure device 101.

The tissue closure device 101 includes an assembled pair of substantially identical closure components 105 and 107. Each of the closure components 105 and 107 includes a tissue attachment base 109 with an attachment mechanism on a first side (e.g., the underside of the tissue attachment base 109). The attachment mechanism is used to affix the attachment base 109 to, without limitation, the skin or other tissue. The attachment mechanism may be an adhesive. The adhesive may be designed such that the user has some time to make minor placement adjustments before the adhesive adheres to the skin surface. This time can range from approximately ten seconds to approximately three minutes, and in a preferred embodiment is approximately one minute. The adhesive may be, without limitation, a pressure sensitive adhesive. In various embodiments, the attachment mechanism may include one or more hooks to affix the attachment base to the skin or other tissue.

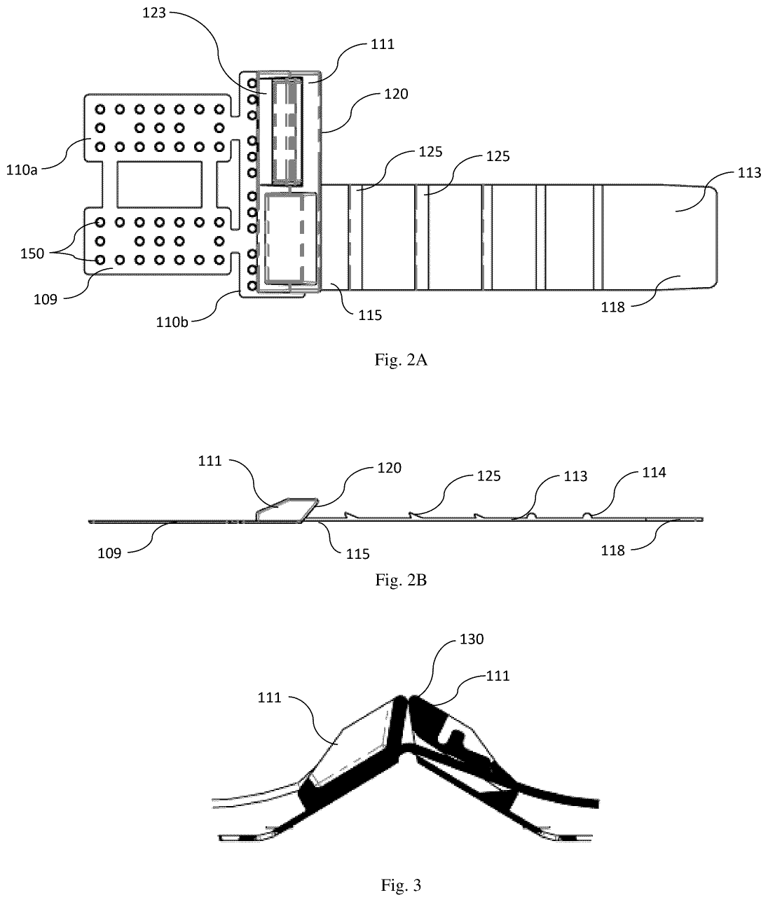

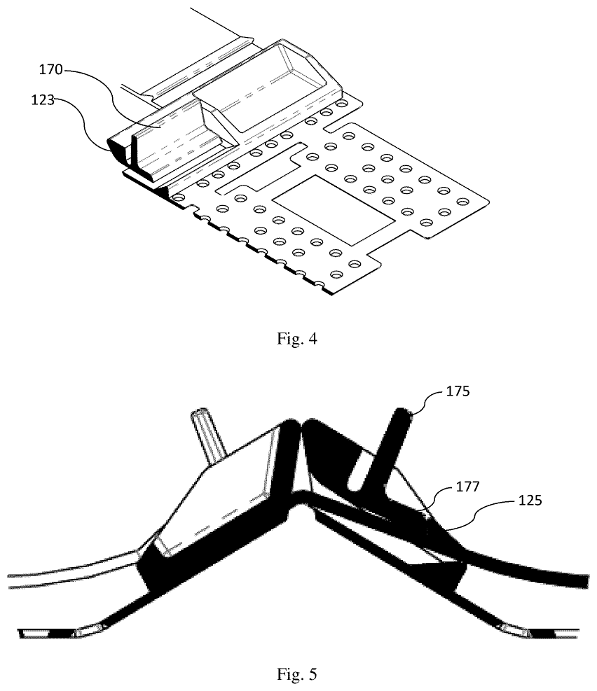

FIGS. 2A and 2B depict top and side views of a single closure component. A standoff assembly 111 is mounted on a second side of the attachment base 109 of each closure component. Preferably, the attachment base 109 is comprised of two portions, an anchor 110a and a wing 110b. Preferably wing 110b is adapted to be positioned adjacent to the wound edge while anchor 110a is adapted to be positioned away from the wound edge and provide additional adhesion to the skin. Anchor 110a is preferably coupled to wing 110b by one or more living hinges or flexible portions of base 109. However, anchor 110a may be integrated into wing 110b or otherwise attached to wing 110b. Anchor 110a is shown as having two portions attached by bridges and is narrower than wing 110b. However, anchor 110a can be a continuous piece, be a single piece (as shown in FIGS. 4A and 4B), be two or more separate pieces, have another number of bridges, and/or expand the full width of wing 110b.

The whole standoff assembly preferably encompasses the entirety of the wing. The wing is what, with the adhesive, attaches to the wound's edges and pulls the edges up, everting the wound edges as the device is closed. The wing is also what maintains the approximation of the wound's edges during closure. By creating the standoff assembly across the entirety of the wing, a uniform tension (pressure) is exerted on the closed, everted wound.

A pull-tab 113, having first and second ends 115 and 118, respectively, is coupled to the attachment base 109 through the standoff assembly 111 and defines a longitudinal axis. The standoff assembly 111 has a forward face 120, from which the first end 115 of the pull-tab 113 extends, and an opposed rearward face. The forward face 120 includes a sloped portion that is sloped rearward as it approaches the attachment base 109.

Standoff assembly 111 also includes a female connective element 123. The female connective element 123 receives and engages the pull-tab 113 from the other component. When the closure components 105 and 107 are engaged, the pull-tab 113 of each closure component 105 and 107 member passes through the female connective element 123 of the other member. The pull-tab 113 has teeth 125 rising from its upper surface. Upon entry of a pull-tab 113 into its associated female connective element 123, the female connective element 123 prevents teeth 125 from unintentionally disengaging from standoff assembly 111. As described in further detail below, the female connective element 123 may include a releasable tab-locking mechanism. Pull-tab 113 may also include grip enhancing elements. For example, as shown in the figures, pull-tab 113 may include bumps or projections 114. In other embodiment, pull-tab 113 may have knurling or other texture applied to increase friction, may have a friction increasing coating and/or may have holes or divots to increase gripability.

FIG. 3 depicts a close-up, cut-away view of standoff assemblies 111 in the engaged position. As the pull-tabs 113 are pulled through female connective element 123 of the other closure component 105 and 107, the closure components 105 and 107 and hence the edges of the tissue opening to which the attachment base 109 is adhered to, move closer to each other. Further pulling brings the sloped portions of the forward faces 120 of the closure component components 106 and 107 into substantial contact with one another at a location of contact 130, whereupon each standoff assembly 111 pivots about the location of contact 130. When this occurs, respective portions of the undersurfaces of each tissue attachment base 109, which have adhesive covering and which are adhered to the skin or other tissue during use, are lifted, inducing eversion of the tissue edges that define the tissue opening. To facilitate wound edge eversion, the tissue closure device preferably has pull-tabs 113 in the lower one-third of the height of each standoff assembly 111, and preferably has locations of contact 125 in the top one-third of the height of each standoff assembly 111.

The attachment base 109 of the closure devices 105 and 107 may be porous to allow for tissue breathing and the egress of tissue fluids. Human tissues, unlike many other mammalian species, respire, produce oils and moisture. These physiologic processes may result in premature dislodgement and migration of the device 101 and thereby render the device 101 ineffective. This may be overcome by making the attachment base 109 porous. For example, the attachment base 109 may include porous material, and/or a critical mass of one or more predefined holes 150, that allow for tissue breathing and the egress of tissue fluids and/or gasses without effecting the adhesive performance of the device 101.

It is preferred that the tissue opening to be treated is smaller than the length of the pull-tabs. However, even for a tissue opening larger than the length of the pull-tabs, the members can be applied to both sides of the opening and manually brought together to allow the pull-tabs to engage with the respective openings on paired members. A further benefit of certain embodiments of the invention is that wounds can be closed over the course of multiple application steps. The person applying the tissue closure devices can choose to engage the members and close the tissue opening either partially or fully. Accordingly, the device preferably has at least one tooth 125 to accomplish complete engagement of the device in the closed position but may have a total of two or more teeth to allow a partial closure or a series of partial closures reducing edge separation until full closure and edge eversion is accomplished by engaging the last tooth. In some embodiments the teeth are all the same size, while in others the teeth may have different heights.

In a preferred embodiment, a non-locking tooth is of a height inferior to the height of a locking tooth but of a sufficient height to be able to hold enough tension to prevent the device from fully opening if the locking tooth is released from the locking mechanism after closure. In a further embodiment, one or more additional teeth may be positioned along the length of the tab of interior height to the locking tooth capable, once engaged by the locking mechanism, of maintaining the degree of openness of device if the locking tooth is not engaged so that the device can be used for staged closure of a wound. The teeth along the length of the tab engage the locking mechanism sufficiently to allow the tooth to pass through while allowing the locking mechanism to return to its prior position to hold the tooth in place and not allow the engaged tooth to move backwards thereby preventing the device from reopening.

The locking mechanism may have a portion adapted to engage the locking tooth, or an inferior tooth in a staged closure, to assure that once engaged by a tooth of the device, it will not release the tooth and allow the device to open. Such locking mechanism may be levered so that it is capable of being forced open sufficiently to allow the tooth to be engaged to pass, and each successive tooth until the locking tooth is engaged, and at the same time, maintaining sufficient tension against the tab behind the engaged tooth to ensure that the device does not open after engagement of the tooth.

Preferably, standoff assembly 111 is equal in width wing 110b. Preferably, the greater portion standoff assembly 111 covers of wing 110b the greater the stability of device 101 upon application to a wound. Larger standoff assemblies 111 preferably reduce torsional forces experienced by smaller standoff assemblies 111. Preferably, pull-tab 113 is about 90% the width of standoff assembly 111, about 95% the width of standoff assembly 111, or about 98% the width of standoff assembly 111.

As shown in FIG. 4, the female connective element 123 of each closure device 105 and 107 may have a releasable tab-locking mechanism 170 configured to releasably lock the pull-tab 113 in position. Such a locking mechanism may be unlocked by a tool, for example, tweezers or a needle, or in some embodiments a human finger, and the lock can subsequently be reengaged on the pull-tab to relock the pull-tab. The releasable tab-locking mechanism 170 advantageously allows for drainage of wound edema and/or the treatment of infection, without removal and reapplication of the device. Such a feature can also be helpful when a tissue opening needs to be temporarily left open to facilitate healing.

FIG. 5 depicts a close-up, cutaway view of releasable mechanism 170 in the engaged position. Preferably, the releasable mechanism is configured to allow the locking mechanism to release the tooth on the tab utilized to maintain the device in its state of lock status. The releasable locking mechanism 170 may have a finger 175 to disengage the locking mechanism. The finger 175 may be, for example, positioned in a recess, such that a tool is required to manipulate the finger 175. In other embodiments the finger 175 may protrude from the recess such that it can be manipulated without a special tool. Releasable tab-locking mechanism 170 may have a tip 177 that can engage against the teeth 125 the pull-tab 113. For example, in the unreleased position, the tip 177 may engage against a tooth 125 on the pull-tab, with the finger 175 being biased in the unreleased position. By rotating the finger 175, a released position is obtained wherein the pull-tab can be freely moved.

In other embodiments, a ratchet/piston locking mechanism may be provided that allows for precise closure and locking of the paired components. By depressing the piston, the locking mechanism is securely unlatched. In various embodiments, the locking mechanism may include a hollow recess. A tool, for example a needle, can be used to unlock the locking mechanism, for example by engaging the end of the needle into the hollow recess of the locking mechanism, applying force to the locking mechanism through the tool, and with that force unlocking the locking mechanism. The releasable locking mechanism allows for drainage of wound edema and/or the treatment of infection, without removal and reapplication of the device. Such a feature can also be helpful when a wound needs to be temporarily reopened to facilitate healing after closure.

The tissue closure device may be manufactured and provided to an operator in the engaged position, such that the second end already protrudes from the female connective element of the other closure component. Alternatively, the tissue closure device may be provided to an operator in the unengaged position, with the second end of each pull tab being dimensioned and/or having enough flexibility so as be capable of being passed through the female connective element of the other closure component.

Embodiments of the invention may be applied as follows. Assembly of the pairs of closure components that make up the closure device may be done prior to packaging and before the user receives the devices. Assembly may be done by pushing the pull-tabs of each closure component through the female connective element of its corresponding closure component, to initiate alignment and engage the paired closure devices. Adhesive backing may be applied to the tissue attachment base of each closure device, and an adhesive strip may be applied across the paired closure device to maintain proper alignment.

To apply the assembled closure devices to the tissue opening, the user may remove the adhesive backing from one closure component of the tissue closing device, or, in the case of multiple sets of paired closure components, from all closure components on one side of the engaged sets. The user may then place the closure component(s) with the adhesive backing removed on cleansed and dry skin so that their forward face(s) are abutting one edge of the tissue opening. The user then slowly brings the paired closure components together, one closure component of each pair attached to the skin on one side of the wound and the other closure component each pair not yet attached to the skin. The user continues to bring the paired connections together until the second component of each paired component is placed abutting an opposite skin edge. The user then removes the adhesive backing from the second component(s) and places the second component(s) near the wound edge. The adhesive may be selected such that the user has some time to make minor placement adjustments before the adhesive adheres to the skin surface. This time can range from approximately ten seconds to approximately three minutes, and in a preferred embodiment is approximately one minute. After this time elapses, the user gently brings the edges of the tissue opening wound edges toward each other. The user can, if desired, unlock the closure component(s) by manipulating the tab-locking mechanism.

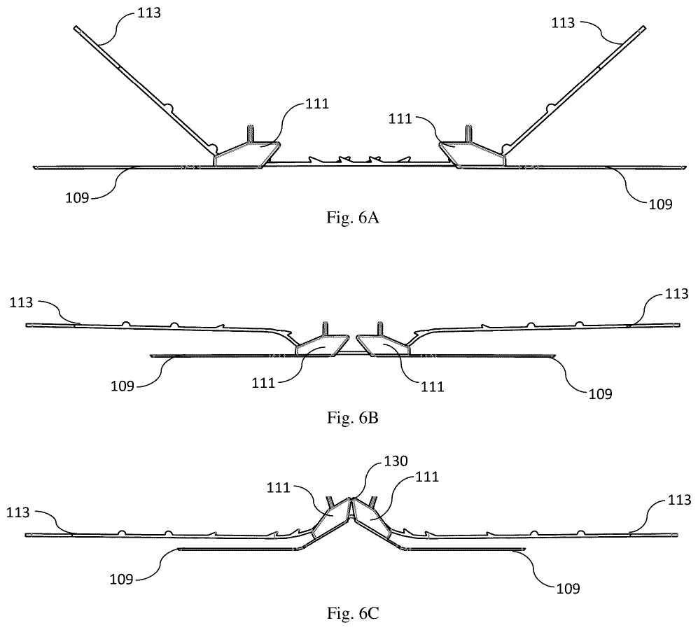

FIGS. 6A-C show several positions of the pair of closure components as they are brought together to close the wound. FIG. 6A depicts the pair of closure components in the engaged and open position. This position allows for the placement of the closure components on either side of the wound. FIG. 6B depicts the pair of closure components as they are drawn together. When a staged closure of the wound is recommended, the position of the closure components depicted in FIG. 6B may be the intermediary or one of several intermediary positions the wound is held at during the process of closing the wound. Preferably, in multi-tooth embodiments, in the position shown in FIG. 6B at least one tooth has been engaged by the locking mechanism. FIG. 6C shows the device in the closed/everted position. Due to the rotation of the standoff assemblies 111 about the contact point 130, the device pulls the edges of the skin upwards thereby everting the wound. Preferably, in the position shown in FIG. 6C the final or sole tooth is engaged by the locking mechanism.

Depending on the type of adhesive used, the closure device will naturally exfoliate within a period of at least approximately seven to ten days for longer duration closures. For other closures, the time may be shorter or longer. Alternatively, a user can apply a solvent to the adhesive for more immediate removal. If desired, the paired closure components can be loosened a variable distance, for example from approximately 1 mm to approximately 10 mm to allow for drainage of fluids and making the window a period of up to 14 days. The paired closure components can later tightened to re-close the tissue opening.

Preferably, skin closure device 101 applies full support to the closed wound. The device is adapted to provide a uniform pressure of closure across the supported length of the wound. The device has a closing structure that preferably fully encompasses the length of wound being closed. For larger wounds, due to the standoff assemblies spanning the entirety of the wings, multiple closure components can be positioned directly adjacent to one another without gaps between the standoff assemblies. In such a configuration, a wound larger than the closure component can be closed while still maintaining uniform pressure along the wound edges. The device preferably has two sides of a closing structure that are actuated by the closing mechanism.

The device works by pulling the edges of the wound together and everting them on closure, after which the device is locked. The device differs from other similar devices in that the closure mechanism expands across the entirety of the closure area, meaning that there is a unform pressure of closure across the entirety of the device. As a result, the device is able to apply a consistent and uniform pressure of closure across the entirety of the area closed.

It was surprisingly discovered that continuous, evenly distributed pressure to the wound helps to remove excess fluid, debris, and bacteria, while also stimulating new tissue growth. This process optimizes the physiological conditions for healing and can lead to faster wound closure and reduced risk of infection. The resulting uniformity of the contact of the live skin of the underside of the wound's edges that are everted creates a uniform structure for healing. The uniform pressure not only promotes conformity but also reduces motion of the wound. This reduction in the motion of the wound arising out of the continuous evenly, distributed pressure occurs both during and after wound closure. Upon the top of the clips meeting as a part of the process of closing of the device, the supported structure of the device prevents any torsioning of the wound during closure. This ensures that the wound itself is closed uniformly, with uniform eversion of the wound edges. The result is the everted live surface edges of the wound are uniformly and evenly joined. The absence of torsioning during closure, unlike any other wound closure mechanism, is a unique feature of the described devices.

The device effects a different healing result from other devices because, as opposed the simply holding the wound closed in an everted position, this device applies a consistent and uniform pressure in holding the wound closed across the entirety of the everted wound, unlike any other device or closure medium effecting an everted closure. This consistent and uniform pressure results from the entirety of the closure, and the wing of the device holding the everted edges of the skin, being supported by a reinforced structure that holds all of the elements of the closure and the closing elements of the device together with a uniform pressure. As a result, the entirety of the healing process across the wound is evenly maintained so that a uniform healing can occur.

Other embodiments and uses of the invention will be apparent to those skilled in the art from consideration of the specification and practice of the invention disclosed herein. Although various exemplary embodiments of the invention have been disclosed, it should be apparent to those skilled in the art that various changes and modifications can be made that will achieve some of the advantages of the invention without departing from the true scope of the invention. For example, elements from one embodiment may be used in another embodiment. These and other obvious modifications are intended to be covered by the appended claims.

1. A wound closure system, comprising:
a pair of closure components, each closure component comprising:
a base, the base having an adhesive first side;
a standoff assembly extending from a second side of the base, the standoff assembly having a pull-tab extending therefrom and a female portion adapted to accept the pull-tab of the other closure component;
wherein a width of the base is equal to a width of the standoff assembly to provide support to the wound along the entire width of the closure components.

2. The wound closure system of claim 1, wherein, upon engagement of the closure components, the components pivot about a contact point to evert the edges of the skin.

3. The wound closure system of claim 1, wherein the base is comprised of a wing portion and an anchor portion and the standoff assembly is positioned above the wing portion.

4. The wound closure system of claim 3, wherein the wing portion and the standoff assembly are of a first width and the anchor portion is of a second, different width.

5. The wound closure system of claim 3, wherein the anchor portion is narrower than the wing portion.

6. The wound closure system of claim 1, further comprising a locking mechanism positioned within the female portion and at least one tooth extending from the pull-tab.

7. The wound closure system of claim 6, wherein the locking mechanism is adapted to engage the at least one tooth of the pull-tab.

8. The wound closure system of claim 7, wherein the locking mechanism is releasable.

9. The wound closure system of claim 8, wherein the locking mechanism is releasable via either a tool or a manipulatable finger.

10. The wound closure system of claim 1, wherein the system is adapted to exert a uniform pressure across the width of the closure components.

11. The wound closure system of claim 1, wherein the closure components are adapted to prevent torsion of the wound.

12. The wound closure system of claim 1, wherein each closure component is a single piece of material.

13. The wound closure system of claim 1, wherein each standoff assembly is comprised of a forward face from which the pull tab extends and a reward face, wherein the forward face is sloped rearward as it approaches the adhesive base.

14. The wound closure system of claim 13, wherein, when a closure component is positioned on opposite sides of a wound and the pull tab of each closure component engages the opening in the standoff assembly of the opposing closure component, as the pull tabs are pulled away from each other, the wound is closed such that the contact of the sloped forward faces of each standoff assembly cause eversion of tissue edges of the wound.

15. The wound closure system of claim 1, wherein the system is non-invasive.
